(19) United States
(12) Patent Application Publication
(10) Pub. No.: US 2026/0232895 A1
(43) Pub. Date: Aug. 13, 2026

(54) WEARABLE MEDICAL DEVICE

(71) Applicant: ForCast Orthopedics, Inc., Denver, CO (US)

(72) Inventors: Jared FORAN, Denver, CO (US); Leo A. WHITESIDE, Chesterfield, MO (US); Reynaldo J. QUINTANA, Menlo Park, CA (US); Peter D. NOYMER, Los Gatos, CA (US); Nicholas R. JOZWIAK, Columbus, OH (US)

(73) Assignee: ForCast Orthopedics, Inc., Denver, CO (US)

(21) Appl. No.: 19/471,094

(22) PCT Filed: Mar. 29, 2024
(86) PCT No.: PCT/US2024/022273

§ 371 Date: Sep. 30, 2025

Related U.S. Application Data
(60) Provisional application No. 63/493,339, filed on Mar. 31, 2023.
(60) Provisional application No. 63/493,344, filed on Mar. 31, 2023.

(51) Int. Cl. A61M 5/142; A61M 39/00; A61M 39/02; A61M 39/24

(52) U.S. Cl. CPC A61M 5/14248; A61M 39/02; A61M 39/24; A61M 2039/0009; A61M 2039/0205; A61M 2202/0007; A61M 2205/12; A61M 2205/3331; A61M 2205/582; A61M 2205/583; A61M 2209/088

(57) ABSTRACT

A wearable medical device and catheter assembly may be provided. In an exemplary embodiment the catheter assembly may include a catheter elbow and a catheter that runs through the catheter elbow, the catheter including an external catheter portion on a first side of the catheter elbow and an internal catheter portion on a second side of the catheter elbow. The assembly may also include a cuff covering a portion of the internal catheter extending from the catheter elbow, a delivery module that interfaces with a pump and is attached to one end of the catheter, and a retention module that retains the delivery module.

### BACKGROUND

Treatment of many medical conditions is challenged by the need to regularly and safely administer medical agents, e.g. antibiotics, chemotherapy or other oncologic therapeutic agents, biotechnology agents, etc. locally at or near the site of the condition to treat over time. One such method of drug delivery is repeated serial local administration (e.g., subcutaneous, intramuscular, intracapsular, intravenous injections) by a clinician. Another method is drug delivery via a reservoir/pump system that allows automatization of the delivery from a reservoir and less frequent administration by a clinician. Furthermore, many diseases are better treated if the therapeutic agent is administered in a manner that is able to achieve a specific concentration or level in the region of interest.

In many cases, a medical agent may put the patient at risk of harmful side effects through systemic exposure. For example, the use of antibiotics to treat a total joint infection, such as PJI (Periprosthetic Joint Infection), fracture-related infections, other implant-related infections, and/or other joint conditions. Under the current standard of care, a PJI patient is typically administered an antibiotic, such as vancomycin, through a PICC (Peripheral Intravenous Central Catheter) line with the intention of increasing the blood serum concentration of the antibiotic to near, but below the safe, AKI (Acute Kidney Injury) limit.

Yet, the joint infection resides within a protective anatomical structure, the joint capsule's synovial barrier, wherein the relative lack of local vasculature inhibits the transfer of antibiotic concentration from the blood stream into the synovial fluid where it is needed to directly treat the infection. The result is that to achieve even modest intra-capsular concentrations, the patient may be exposed to high systemic concentrations which can contribute to other side effects. Meanwhile only a low concentration, often at subtherapeutic levels, can be achieved at the site of infection. Exposure at subtherapeutic levels is considered a primary cause for the development of antibiotic resistance. Without the means of directly applying the antibiotic to the bacterial infection within the joint capsule, the indirect, systemic treatment is suboptimal and may be counterproductive.

Polymeric implants, osmotic pumps and diffusion reservoirs may provide a steady, though declining, rate of drug release for a specific period, some medical agents are more effective when administered in intervals or via bolus dosing over time. Implantable electromechanical infusion pumps can be programmed to deliver drugs at consistent and specific dosages, delivery rates and intervals. These implantable, electromechanical infusion pumps are placed surgically and typically removed surgically when treatment is concluded, or the pump life has been exhausted and must be replaced. Given their complexity, these pumps are expensive and therefore not economically suitable for certain uses, for example treatment of PJI. Further, their complexity also contributes to greater potential for device failure, resulting in a potential for patient injury, and/or the need for more frequent surgical removal and replacement.

Another method of medication delivery that is often used is a port-a-cath in which a catheter is surgically inserted into the synovial cavity and be left embedded for the duration of a treatment. A septum may then be placed underneath the skin allowing for easier access to the interior catheter for delivering or extracting fluids. However, existing port-a-caths may be limited in what they can interface with (i.e., in general, port-a-caths simply provide easier access for a nurse to insert a needle or other related tool).

Surgically inserted catheter systems may often be more economical than implantable pumps. However, often the patient is left with a tube dangling from their skin, which may both inconvenience the patient as well as endanger the patient. Further, with the catheter being unsecured as it exits the body, it has the potential to move or to be moved, thus further expanding or irritating the exit site and exacerbating the risk of infection. Additionally, the catheter may snag or get caught on an object, which may dislodge the catheter entirely and have disastrous consequences for the patient.

### SUMMARY

A wearable medical device and catheter assembly may be provided. In an exemplary embodiment the catheter assembly may include a catheter elbow and a catheter that runs through the catheter elbow, the catheter including an external catheter portion on a first side of the catheter elbow and an internal catheter portion on a second side of the catheter elbow. The assembly may also include a cuff covering a portion of the internal catheter extending from the catheter elbow, a delivery module that interfaces with a pump and is attached to one end of the catheter, and a retention module that retains the delivery module.

### BRIEF DESCRIPTION OF THE FIGURES

Advantages of embodiments of the present invention will be apparent from the following detailed description of the exemplary embodiments. The following detailed description should be considered in conjunction with the accompanying figures in which:

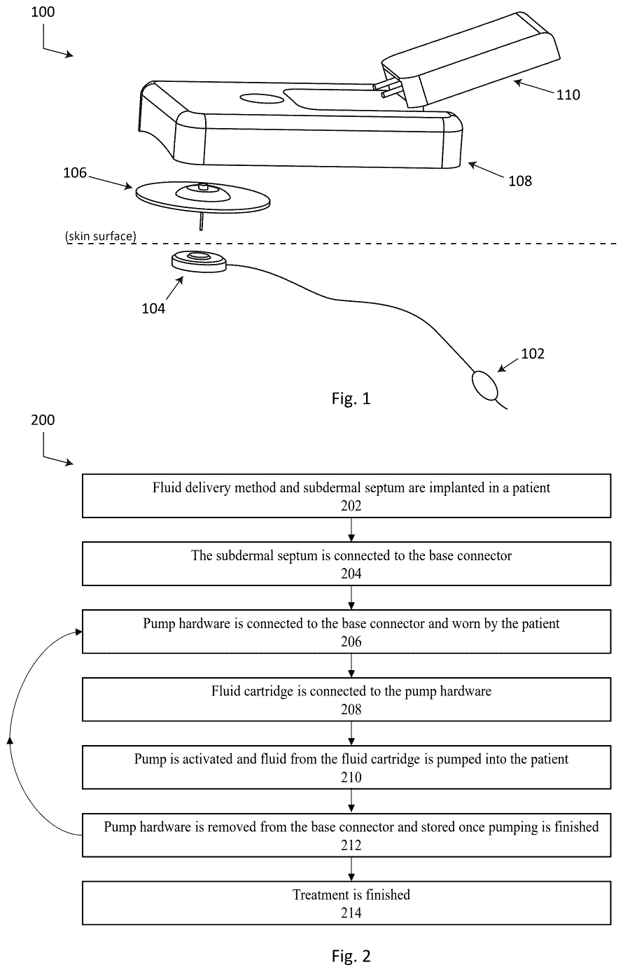

Exemplary FIG. 1 shows an embodiment of a wearable medical device and catheter assembly.

Exemplary FIG. 2 shows an embodiment of a method for using a wearable medical device and catheter assembly.

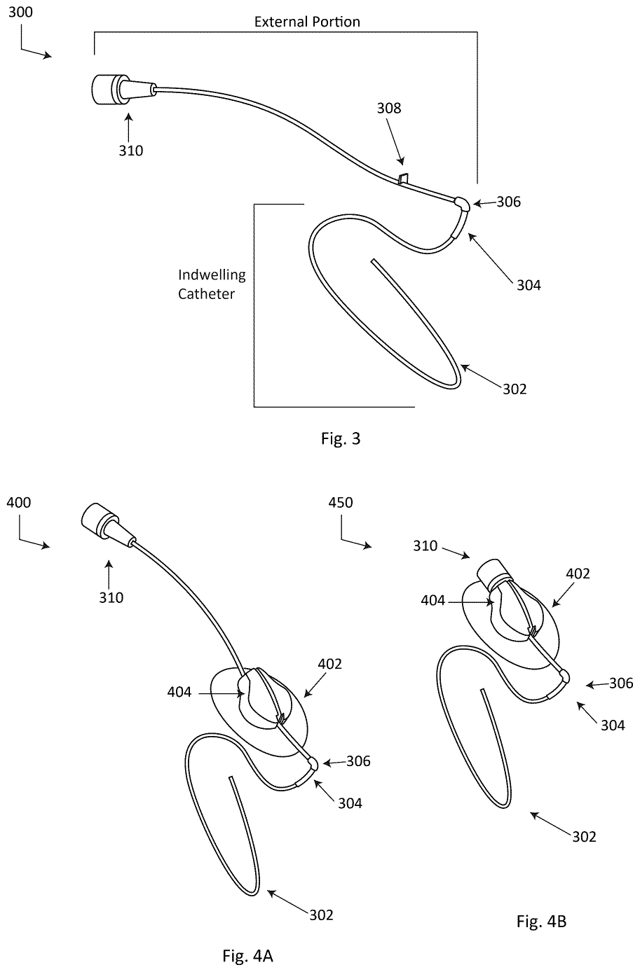

Exemplary FIG. 3 shows a catheter assembly embodiment.

Exemplary FIG. 4A shows an exemplary catheter assembly with a storage module and extended catheter.

Exemplary FIG. 4B shows an exemplary catheter assembly with a storage module and retracted catheter.

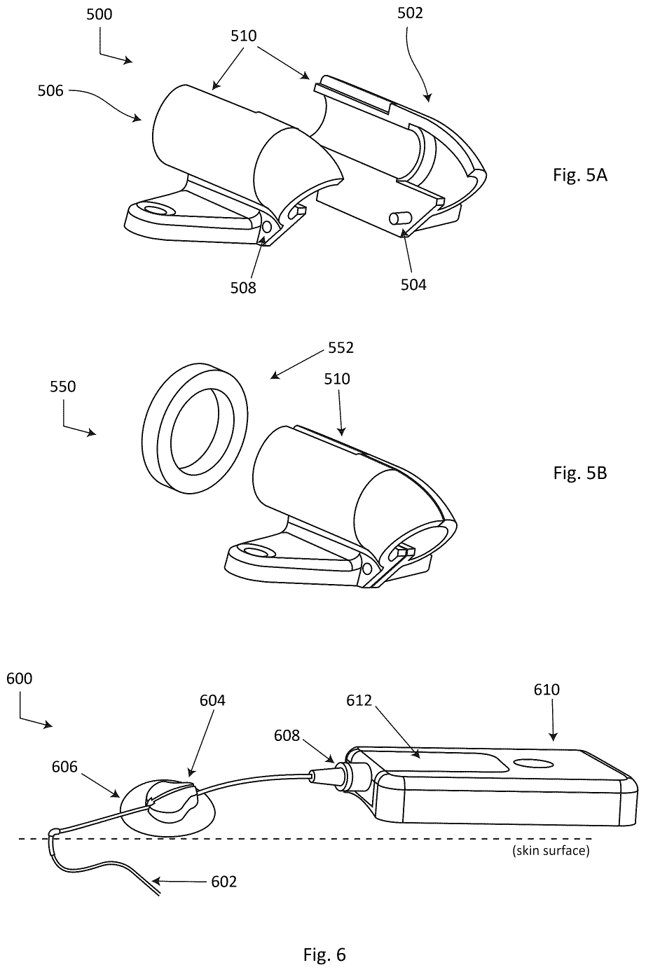

Exemplary FIG. 5A shows a catheter elbow embodiment.

Exemplary FIG. 5B shows a catheter elbow embodiment with a retaining ring.

Exemplary FIG. 6 shows an embodiment of a catheter assembly connected to a wearable medical device.

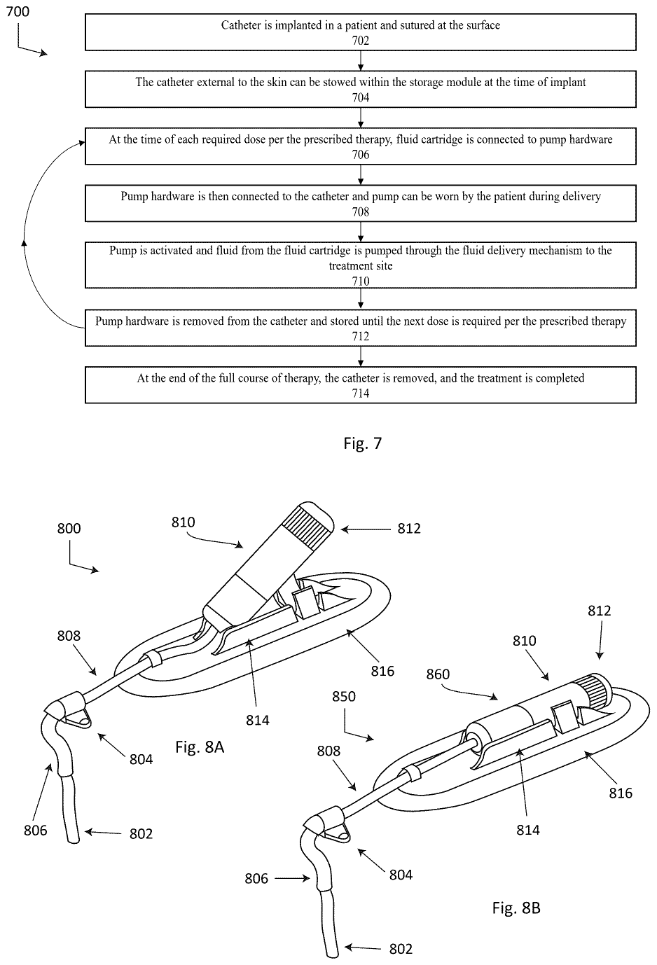

Exemplary FIG. 7 shows an embodiment of a method for using a catheter assembly and wearable medical device.

Exemplary FIG. 8A shows an alternative embodiment of a catheter assembly.

Exemplary FIG. 8B shows another view of the alternative embodiment of a catheter assembly.

### DETAILED DESCRIPTION

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention”, “embodiments” or “invention” do not require that all embodiments of the invention include the discussed feature, advantage or mode of operation. It may be understood that parts of the described embodiments and methods may be implemented in the context of any other embodiment or methods.

In one or more exemplary embodiment a wearable medical device may be provided.

In an exemplary embodiment the wearable assembly may be used to treat, for example, periprosthetic joint infections (PJI). The wearable assembly may include a fluid delivery mechanism. The fluid delivery mechanism may be attached to a subdermal septum. The subdermal septum may rest on the interior of a patient's skin and may be attached to a base connector which may rest on the exterior surface of the patient's skin. The base connector may then be further connected to pump hardware, such as, for example, an infusion pump. In some embodiments the infusion pump may be able to accept fluid cartridges. The fluid cartridge may be filled with, for example, antibiotics, saline flush, a different fluid with clinical benefit, or a combination of fluids such as a mixture of saline and antibiotics.

An exemplary method of using the wearable medical device and catheter assembly may be provided. In a first step, the fluid delivery mechanism and subdermal septum may be implanted in a patient. The implantation may be done during surgery, for example surgical intervention for joint infection such as a washout treatment or a revision treatment. In a next step the subdermal septum, which may rest interior the patient's skin, may be connected to the base connector, which may rest exterior the patient's skin. In a next step the pump hardware may be connected to the base connector and may be worn by the patient. In a next step the fluid cartridge may be connected to the pump hardware. In a next step the pump hardware may be activated and fluid from the fluid cartridge may be pumped through the fluid delivery mechanism to the treatment site. In a next step, once the full dose is delivered, which may be when the fluid cartridge is emptied, when a predetermined amount of fluid has been pumped, and/or when a specified amount of time has passed, pump hardware may be removed from the base connector and stored. In some embodiments the steps may be repeated periodically, for example once per a day, or once every other day, until treatment is finished. In a last step, the treatment may be finished and the fluid delivery mechanism may be removed, for example.

Referring now to FIG. 1 an exemplary wearable medical device and catheter assembly 100 may be shown. In an exemplary embodiment the wearable assembly 100 may be used to treat, for example, periprosthetic joint infections (PJI). The wearable assembly 100 may include a fluid delivery mechanism 102, which may be, for example, catheter with a balloon and catheter tip which may be able to inject fluid into, for example, an infected joint or other portion of the body. The fluid delivery mechanism 102 may be attached to a subdermal septum 104. The subdermal septum 104 may rest interior a patient's skin and may be attached to a base connector 106 which may rest on the exterior surface of the patient's skin. The base connector 106 may further include a cannula or other method of allowing fluid transfer between the base connector 106 and the subdermal septum 104. The base connector 106 may then be further connected to pump hardware 108, such as, for example, an infusion pump, an oscillation pump, a plunger, etc. In some embodiments the infusion pump may be able to accept fluid cartridges 110, in other embodiments the pump may accept medicine or fluid from a different source. The fluid cartridge 110 may be filled with, for example, antibiotics, saline flush, a different fluid with clinical benefit, or a combination of fluids such as a mixture of saline and antibiotics.

Referring now to FIG. 2 an exemplary method of using the wearable medical device and catheter assembly 200 may be shown. In a first step 202, the fluid delivery mechanism 102 and subdermal septum 104 may be implanted in a patient. The implantation may be done during surgery, for example surgical intervention for joint infection such as a washout treatment or a revision treatment. In a next step 204 the subdermal septum 104, which may rest interior the patient's skin, may be connected to the base connector 106, which may rest exterior the patient's skin. In a next step 206 the pump hardware 108 may be connected to the base connector 106, and may be worn by the patient. In a next step 208 the fluid cartridge 110 may be connected to the pump hardware 108. In a next step 210 the pump hardware 110 may be activated and fluid from the fluid cartridge 110 may be pumped into the patient. In a next step 212, once pumping is finished which may be when the fluid cartridge 110 is emptied, when a predetermined amount of fluid has been pumped, and/or when a specified amount of time has passed, pump hardware 108 may be removed from the base connector and stored. In some embodiments steps 206-212 may be repeated periodically, for example once per a day, or once every other day, until treatment is finished. In a last step 214 treatment may be finished, treatment may be finished after a specified period of time, e.g. 6 weeks or 2 months, or after the initial problem, e.g. PJI, has been cured or sufficiently treated. In some embodiments a checkup may be performed after a specified period of time in order to determine whether treatment should continue. It may be understood that after treatment additional surgeries may be performed and the fluid delivery mechanism 102 and subdermal septum 104 may be removed.

An exemplary fluid delivery mechanism 102 may now be described with more detail. The fluid delivery mechanism 102 may be a catheter, such as a port-a-cath, and may include the subdermal septum 104. The fluid delivery mechanism may have a specialized tip for dispensing fluid, for example a balloon tip or a fenestrated tip. In addition, the catheter tube may also have a rigid tube or tip attached to it, e.g., a trocar tip to facilitate placement of the catheter tube. The rigid tip or tube may be easier to place since it does not deform easily like the catheter tube. It may be employed only for placement and may be removed once the catheter is inserted in the desired location. In another embodiment, a guide wire may be used to place the catheter. The guide wire (unlike the rigid tube at the catheter tip) may not be connected to the catheter. The guide wire may first be inserted into the desired location, then the catheter may be placed over the guide wire and pushed through. The softer catheter tube may conform and bend, following the path of the guide wire. Once the catheter is placed in the desired location, the guide wire may be removed. The fluid delivery mechanism 102 and subdermal septum 104 may be specially constructed to interface with specific hardware, such as, for example, the base connector 106. In some embodiments the subdermal septum 104 may include an means for locating and anchoring the fluid delivery mechanism 102, for example a magnet. In some embodiments the fluid delivery mechanism 102 may be anchored to tissue within the patient's body which may allow the base connector 106 and the pump mechanism 108 to be supported by the fluid delivery mechanism 102 when worn by the patient. The fluid delivery mechanism 102 may lead to a specific area in need of treatment, for example an infected joint in the case of PJI treatment. The tip may need to be secured to the treatment area, for example a balloon tip may be used to ensure the dispensing tip is properly secured to a joint in need of treatment. In some embodiments the fluid delivery mechanism 102 may have an adjustable length, for example by being cut to a specific size prior to and/or during surgery. It may be contemplated that in some cases the fluid delivery mechanism 102 may not be entirely within the patient and may be a hybrid system where part of the mechanism is underneath the skin and part of the mechanism comes through the skin in order to connect, directly or indirectly, to the pump hardware 108.

An exemplary base connector 106 may now be described with more detail. The base connector 106 may be inserted into or connected with the subdermal septum 104 following the surgical procedure where the subdermal septum 104 is implanted. In one embodiment the base connector 106 may be a pressure sensitive adhesive on the skin side of a pliable disk or membrane, which may be further covered with a protective element. To apply the base connector 106, the protective element may be removed, and the pliable disk or membrane may be pulled back in order to present a narrow-gauge needle or other connector. The narrow-gauge needle or other connector may then interface with the subdermal septum 104, and the pliable disk or membrane may be adhered to the patient's skin, for example through the pressure sensitive adhesive. In some embodiments the connector may be a cannula with Luer-lock fittings to interface with the pump.

In some embodiments the base connector 106 may need to be replaced periodically, for example every week, every day, every 3 days etc. The changing may be done by the patient or a clinician. depending on the specifics of the treatment being performed. The base connector 106 may contain guidance features that assist in connecting the base connector 106 to the subdermal septum 104, for example the subdermal septum 104 may have a conical shape with a flat end instead of a pointed one and once implanted this may result in a slight conical protrusion of the patient's skin. The base connector 106 may then have an equivalent indentation which may match with the protrusion on the patient's skin, thereby helping visually identify where the base connector 106 should be placed and then helping in the actual placement of the base connector 106. In this embodiment the needle may be along the perimeter of the base connector rather than centered, which may help avoid repeatedly puncturing the skin in the same place, and therefore, may be more comfortable/less harmful to the patient in the long term. Each time the base connector 106 is replaced it may be rotated slightly allowing for a different location to be punctured.

In an exemplary embodiment the base connector 106 may be directly integrated into the pump mechanism 108 and may be applied and removed each time the pump is used. In some cases, this may reduce the probability of an infection occurring.

An exemplary pump mechanism 108 may now be described with more detail. In some embodiments the pump mechanism 108 may be able to snap or bind to the base connector 106. For example, snaps and detents may be used to guide the connection and in some embodiments buttons may be used to disconnect after fluid delivery is complete. In other embodiments the pump mechanism 108 may be magnetically connected coupled to the base station 106, for example by one component having holes and the other having short magnetic pins that correspond to the holes, the magnetic strength may be enough to ensure a firm coupling without being so strong as to create excessive stress on the fluid delivery mechanism 102. In yet other embodiments any other connection method known in the art may be used, or may be a combination of methods, e.g. a mechanical interface combined with magnetic coupling. In an exemplary embodiment the fluid delivery mechanism may be partially external to the skin and may connect directly to the pump mechanism through, for example, a Luer-lock fitting, and therefore the base connector 106 may not be needed.

In some embodiments the pump mechanism 108 may be connected to the base connector 106 before the pump mechanism 108 is attached to the subdermal septum 104. Connection with the pump mechanism 108 may allow the base connector 106 to receive electric power and may enable electronic guiding features on the pump mechanism 108 to assist in connecting the base connector 106 to the subdermal septum 104. The electronic guiding features may include, for example, a plurality of feedback signals, scanning capability, etc. In an exemplary embodiment the pump mechanism 108 may include, for example, a display. The display may work by, for example, displaying the location of the subdermal septum 104 and once the needle from the base connector is aligned may alert the user, through audio, visual, and/or tactile notifications.

The pump mechanism 108 may further contain information related to, for example, the progress of fluid delivery, battery status information, and relevant sensor information, etc.

An exemplary fluid cartridge 110 may now be described with more detail. In some embodiments a dual-reservoir cartridge may be used. The pump mechanism 108 may be configured to deliver from the dual-reservoir cartridge by, for example, delivering from each reservoir sequentially. Each reservoir may contain a different fluid or mixture of fluids, for example one may contain and antibiotic or other medicine and the other may contain saline or a saline mixture. It may be contemplated that the reservoirs may be different sizes, for example the saline reservoir may be 5-10 ml while the medicinal reservoir is 3-5 ml, though in other embodiments they may be the same size.

In other embodiments a single reservoir may be utilized, if multiple fluids need to be injected then the entire fluid cartridge 110 may be replaced with a new fluid cartridge 110 that contains the second fluid. In some embodiments a single fluid may be injected through the pump mechanism while a saline flush or other cleaning methodology may be utilized by, for example, applying a prefilled syringe directly into the base connector 104.

In an embodiment the fluid cartridge 110 may have one or more syringe type reservoirs, which may be driven by, for example, a motorized lead screw to move the syringe plunger. In other embodiments the plunger could instead be driven by, for example, a compressed spring. If there are two or more reservoirs each reservoir may require its own driving mechanism. In a different embodiment an elastomeric container with a single exit with a check valve which only allows for the egress of fluids may be used. Pumping from the elastomeric container may be done by, for example, oscillating the container such that the check valve is forced open and some or all of the fluid is ejected, after the oscillation is completed the check valve may then close preventing re-entry of fluids. In other embodiments any other method known in the art may be used to dispense the fluid, for example using bellows.

In some embodiments the amount of fluid injected into the patient may be controlled by the amount of fluid contained in the fluid cartridge 110. Therefore, operation may be simplified for the user and adjustments may be made by changing to a different size or type of fluid cartridge 110.

In some embodiments the fluid pathway from the fluid cartridge 110 to the area being treated may be sterile at the time of each use, which may mean that for each fresh cartridge, none of the formulation comes into contact with any reusable parts of the pump. This connection may be aided by having a sufficiently close fit of the fluid cartridge 110 to the pump mechanism 108. In some embodiments the sterility may be enforced by the fluid cartridge 110 having a short catheter line with a Luer-lock or similar feature that allows for connection to the base connector 106. This pathway may also be used as a backup if, for example, the connection of the cannula to the fluid delivery mechanism 102 is determined to be too flimsy.

In some embodiments where an antibiotic is used the antibiotic may be stored as lyophilized powders due to relatively short shelf life when reconstituted in an aqueous solution. In these cases, the fluid cartridge 110 may contain two or more chambers with a membrane/barrier between the chambers. In some embodiments one or more chambers may contain a powder while one or more other chambers contain a diluent, the barrier may be breakable by the patient or a nurse/other medical professional which may allow the powder to combine with the diluent to create a medicinal fluid.

In another embodiment two individual syringes may be connected via a manually operated valve, where the first syringe contains the powder and the second syringe contains the diluent. The syringe with the powder may have enough excess space to hold a resulting mixture of the powder and diluent, and the mixing may be initiated by the patient or another person manually opening the valve between the two syringes, which may then be, for example, inserted into the fluid cartridge 110.

In an exemplary embodiment a liquid tight connection between the reservoir or reservoirs and the base connector may be required. In an exemplary embodiment the connection may be formed with, for example, a Luer-lock. In another embodiment the connector may be a rigid tube that protrudes from the fluid cartridge 110, for example the reservoir may be connected to a soft elastomeric cylinder with a hole through its axis, the diameter of which is slightly larger than the tube around which it will seal. In this embodiment all surfaces of the soft elastomeric cylinder except the inside diameter may be constrained, which may allow for the material to bulge into the unconstrained inside diameter when a compressive force along the cylindrical axis is applied. If the rigid tube is placed into the inner hole of the soft elastomeric cylinder prior to applying the pressure, the inside diameter of the cylinder may reduce and clamp to the rigid tube's outer walls. This way a tube from the container may be connected to a tube on the back side of the pump mechanism 108 in a liquid tight manner.

Referring to FIG. 3, an exemplary catheter assembly 300 may be shown. The catheter 300 may enable the delivery of drugs, saline, and/or other clinically beneficial fluids to a treatment area in a patient, for example to an infected joint space. The catheter 300 may have a catheter tube 302, part of which may be inserted inside a patient and part of which may be external to the patient when in use. The implantation may be done during surgery, for example surgical intervention for joint infection such as a washout treatment or a revision treatment. The catheter tube 302 may be a continuous pliable tube, and may be made of, for example, flexible polymer, silicon, polyvinyl chloride (PVC), pellathane (TPU), isoplast (ETPU), polypropylene, etc. In some embodiments the length of the catheter tube 302 may be adjustable, which may include, for example, cutting the catheter tube 302. In some embodiments the end of the catheter tube 302 may have a specialized tip for dispensing fluid to a particular location, for example a balloon tip may be used to hold the catheter tube 302 in place in the synovial cavity. In other embodiments a different tip may be used with or without the balloon tip, for example a fenestrated tip, or the catheter tube may have a rigid tube or tip attached to it, for example, a trocar tip to facilitate placement of the catheter tube. The rigid tube is easier to place since it does not deform easily like the catheter tube (i.e., it may be employed only for placement and may be removed once the catheter is inserted in the desired location). In another embodiment, a guide wire may be used to place the catheter. The guide wire (unlike the rigid tube at the catheter tip) may not be connected to the catheter. The guide wire may first be inserted into the desired location, then the catheter may be placed over the guide wire and pushed through. The softer catheter tube may then conform and bend, following the path of the guide wire. Once the catheter is placed in the desired location, the guide wire is removed.

The catheter 300 may also have a catheter cuff 304 which may be located at least around the area of the catheter tube 302 that exits from the internal area of the patient to external the patient. The catheter cuff 304 may be made from a material that promotes skin and subcutaneous soft tissue wound healing and/or may include adhesive elements to enhance skin closure around the cuff 104, which may reduce the risk for infections arising at the skin/catheter interface. The cuff 304 may be made of materials including, but not limited to, polyester materials such as Dacron, and/or metal mesh materials such as titanium or stainless steel, and in an exemplary embodiment may be a woven or braided sleeve. In another embodiment the cuff 304 may be a polymer extrusion that enhances healing and adhesion.

The catheter may also have a catheter elbow 306. The catheter elbow 306 may allow the catheter tube 302 to exit the patient's body at a specified angle, for example the catheter tube may be set to exit the patient's body at a 90-degree angle which may enable the catheter 300 to maintain a lower profile and reduce the chance the catheter tube 302 snags on external objects. The catheter elbow 306 may further ensure that the catheter tube 302 doesn't kink as a result of a sharp bend, as kinking may reduce the ability of the catheter tube 302 to transport fluids. The catheter elbow 306 may also expose more area of the cuff 304, which may allow for more liberal swabbing, cleaning, and easier application of disinfectants and/or ointments. In an exemplary embodiment the catheter elbow 306 may have one or more suture points which may be applied at the time the catheter 300 is implanted in order to secure the catheter 300 to the patient's skin. The catheter elbow 306 may be designed to be split so that it may be readily removed from the catheter 300, which may facilitate removal of the implanted portion of the catheter 300 at the end of the patient's treatment. For example, in an exemplary embodiment a tool may be used to split and remove the catheter elbow 306, a coring tool may then be used to cut the skin and soft tissue around where the catheter tube 302 exits the patient, and the catheter 300 may be removed from the patient. In some embodiments the catheter tube 302 may also be cut so the coring tool can fit over the catheter tube 302 for the removal process.

The catheter may also have a catheter tube collar 308, which may be anchored to the catheter tube 302, and may be on the external portion of the catheter tube 302. The catheter 300 may further have a connector assembly 310, which may connect the catheter tube 302 to, for example, a pump or a syringe. The connector assembly 310 may be on the external end of the catheter 300, and may connect the catheter 300 to a pump, cartridge, or other mechanism, and may provide a way to cap the catheter 300 when the catheter 300 is not in use. In some embodiments the connector assembly 310 may include a check valve, which may only allow fluid to enter the catheter 300, and therefore not flow in the opposite direction to the pump, and may assist in mitigating fluid loss if, for example, the patient forgets to cap the catheter 300 after use. In an exemplary embodiment the connector assembly 310 may include, for example, a Luer-lock with a cap. The cap may include a magnet to enable a positive connection between the cap and another component, for example a storage module. In some embodiments the cap may be tethered or otherwise connected to the connector assembly, which may prevent losing the cap. In other exemplary embodiments different methods to form a liquid tight connection between the catheter 300 and a pump or other device may be used, for example an o-ring seal, a needle and septum connection, any other method known to someone of ordinary skill in the art, or a combination of methods.

In an exemplary embodiment the connector assembly 310 may have one or more of the following features: a way to mitigate fluid loss out of the catheter 300 when not connected to a pump or other device; a cap that provides a visual or tactile feedback when placed; a liquid tight seal between the catheter 300 and the pump or other device; and easy connection and disconnection of the pump or other device to the catheter 300.

Referring now to FIGS. 4A and 4B, an exemplary catheter assembly with a storage module and extended catheter 400, and an exemplary catheter assembly with a storage module and retracted catheter 450 may be shown. The assembly 400 may include an adhesive patch 402, which may be applied to the patient's skin and may hold, for example, a storage module 404. In some embodiments the adhesive patch 402 may be replaceable, such as in the case where the adhesive weakens or the patch 402 becomes soiled or dirty. For example, the patch may be replaced every 3-5 days or once a week, additionally in some embodiments the placement site may be changed each time the patch is replaced, which may help mitigate skin irritation. In one embodiment the adhesive patch 402 may be a pressure sensitive adhesive placed on a pliable disk and covered with a protective element. In this embodiment the storage module 404 may be applied by removing the protective element and applying pressure to adhere the adhesive patch 402 to the patient's skin, the storage module 404 could then be attached to the adhesive patch 402, for example via snaps, magnetic coupling, a bayonet fitting, and/or hook and loop fasteners. In other embodiments the storage module 404 may be strapped to the patients arm or leg, for example via a hook and loop fastener. Once the storage module 404 is fastened, the patient or another individual may engage the catheter tube collar 308 into the storage module 404, which may help secure the catheter tube 302 to the storage module 404 and may mitigate stresses and movement of the catheter tube 302 at the skin penetration site.

The storage module 404 may be a housing in which the external portion of the catheter tube 302 may be stored when not in use. In an exemplary embodiment the storage module 404 may be a passive element without any moving parts, in other embodiments the storage module 404 may include active parts that implement various features. For example, the storage module 404 may include a mechanism to forcibly retract the catheter tube 302 or may contain a hinged lid, which may allow the entire external portion of the catheter tube 302 and connector assembly 310 to be stored in the storage module 404 thereby reducing the risk of accidental snagging. In some embodiments the storage module 404 may have rounded edges which may reduce snagging of the storage module 404 itself. The storage module may be applied to the patient's skin before, during, or after the implantation of the catheter 300.

The storage module may further be connected to the catheter tube collar 308, and in an exemplary embodiment the catheter tube 302 may be removed from the storage module 404 by disengaging the catheter tube collar 308.

Referring now to FIGS. 5A-5B, an exemplary two-part catheter elbow 500 and an exemplary catheter elbow with retaining ring 550 may be shown. In an exemplary embodiment the catheter elbow 306 may be comprised of two halves. A first half 502 may have a pin 504 protruding from it that corresponds to a hole 508 in a corresponding place in a second half 506, which may join the two halves together when aligned and pressure is applied. When assembled the catheter elbow 306 may have a small upper groove 510 which may allow a tool, for example a flat head screwdriver, to be used to split the catheter elbow 500 back into two halves. In some embodiments the two halves may instead both have a hole 508, and the two halves may be secured via, for example, a self-tapping screw and/or a bolt and nut. Furthermore, one of the holes may be threaded, which may allow for securing via just a bolt. In an alternate embodiment the two halves may instead be joined by, for example, a self-tapping screw, a bolt and nut, bolt and threaded hole, or an adhesive such as glue or solvent bonding. In yet another embodiment a retaining ring 552 may be used to conjoin the two halves. The retaining ring 552 may be comprised of, for example but not limited to, fabricated metal, a metal or other material extrusion, or an injection-molded polymer. In some embodiments the retaining ring 552 may be cut with diagonal cutters to separate the two halves. In some embodiments instead of a retaining ring the two halves may be adhered or glued together.

Referring to FIGS. 6-7 generally, FIGS. 6 and 7 may show an exemplary embodiment of a wearable medical device and method for use of a wearable medical device which may implement the catheter described above. It may be understood that the catheter described above may be implemented in other embodiments and/or with other medical devices, and the below is for exemplary illustration only.

Referring now to FIG. 6 an exemplary catheter assembly connected to a wearable medical device 600 may be shown. In an exemplary embodiment the catheter assembly 600 may be used to treat, for example, Periprosthetic Joint Infections (PJI). The catheter assembly 600 may include a fluid delivery mechanism 602, which may be, for example, a balloon and catheter tip which may be able to inject fluid through a catheter into, for example, an infected joint or other portion of the body. The catheter tip may be placed at the desired delivery site via, for example, a trocar or guide wire, both of which may be removed once the catheter tip is correctly placed. The fluid delivery mechanism 602 may be attached to a storage mechanism 604 on the exterior of the patient's skin. The storage mechanism 604 may be able to accommodate the portion of the fluid delivery mechanism 602 that is external to the patient's skin when the fluid delivery mechanism 602 is not in use. The storage mechanism may be attached to the patient's skin via, for example, an adhesive patch 606.

At the end of the fluid delivery system 602 there may be a base connector 608. The base connector 608 may then be further connected to pump hardware 610, such as, for example, an infusion pump. In some embodiments the pump hardware 610 may be able to accept fluid cartridges 612, in other embodiments the pump may accept medicine or fluid from a different source such as a syringe. The fluid cartridge 612 may be filled with, for example, antibiotics, saline flush, a different fluid with clinical benefit, or a combination of fluids such as a mixture of saline and antibiotics.

Referring now to FIG. 7 an embodiment of a method for using a catheter assembly and wearable medical device 700 may be shown. In a first step 702, the catheter assembly 600 may be implanted in the patient and sutured at the surface. In a next step 704, the portion of the fluid delivery mechanism 602 external to the patient's skin may be stowed within the storage module 604 at the time of implant. In a next step 706 at the time a dose is required by the patient, the fluid cartridge 612 may be connected to the pump hardware 610. In a next step 708, the pump hardware 610 may be connected to the catheter assembly 600 via the base connector 608, and the pump 610 may be worn by the patient during fluid delivery. In a next step 710, the pump hardware 610 may be activated and fluid from the fluid cartridge 610 may be pumped through the fluid delivery mechanism 602 to the treatment site. In a next step 712, once pumping is finished (i.e., which may be when the fluid cartridge 610 is emptied, when a predetermined amount of fluid has been pumped, and/or when a specified amount of time has passed), pump hardware 608 may be removed from the base connector and stored. In some embodiments, steps 706-712 may be repeated periodically (e.g., once per a day, or once every other day) until treatment is finished. In a last step 714, treatment may be finished after, for example a full course of therapy; after a specified period of time (e.g., 6 weeks, 2 months), or after the initial problem (e.g.,. PJI) has been cured or sufficiently treated. In some embodiments a checkup may be performed after a specified period of time in order to determine whether treatment should continue. It may be understood that after treatment, additional surgeries may be performed and the catheter system 600 may be removed.

Referring to FIGS. 8A-8B, an alternative catheter assembly in a deployed position 800 and an alternative catheter assembly in a locked position 850 according to an embodiment may be shown and described. The catheter assembly 800 may include an indwelling catheter 802 which may be implanted into a patient and lead to, for example, an infected joint space. It may be understood that when in use the indwelling catheter 802 may be placed such that it is inside a patient. The indwelling catheter 802 may connect with a first side of a catheter elbow 804, which may be understood to be at the skin surface of a patient. The catheter elbow 804 may further be connected to a cuff 806 which may cover a portion of the indwelling catheter 802, for example the cuff 806 may be about 15 to 30 mm long. It may be understood that the cuff 806 may allow for tissue ingrowth. A second side of the catheter elbow 804 may further connect to an external catheter portion 808 which may feed into a delivery module 810. It may be understood that in some embodiments the indwelling catheter 802 and external catheter 808 may be two parts of the same catheter piece that continuously runs through the catheter elbow 804, while in other embodiments the indwelling catheter 802 and external catheter 808 may be separate catheters that are attached to each side of the catheter elbow 804 in a fluid tight fashion, for example being glued. The delivery module 810 may include, for example, a cap 812 that may protect the site when not in use and an infusion line connector, which may be a Luer-lock connector in an embodiment. In some embodiments the cap 812 may include a magnet and/or lock threads, which may secure the cap 812 to the retention module 814. The delivery module 810 may be held in a retention module 814 which may be held to the patient, for example by an adhesive patch 816, which may be a pressure sensitive adhesive patch. The retention module 814 may be attached to the adhesive patch 816 via, for example, hook and loop pads or patches. In some embodiments the cap 812 may be tethered to the retention module 814 to prevent loss.

It may be understood that in some embodiments the indwelling catheter 802 and external catheter 808 may be a single catheter that passes from being implanted in the patient to external the patient via the catheter elbow 804. In an embodiment the catheter may be a continuous pliable tube and may have a diameter of about 1.0 to 3.0 mm. The cuff 806 may be made from a material that promoted skin and would healing and provides adhesion to enhance skin closure around it, which may be understood to decrease the risk of infections. In some embodiments the cuff 806 may be made from, for example but not limited to, polyester materials such as Dacron, and/or metal mesh materials such as titanium or stainless steel. In other embodiments the cuff 806 may instead be a polymer extrusion.

The catheter elbow 804 may ensure the catheter exits the body with a tight bend, which may prevent the catheter from accidental snags and/or prevent kinking. The catheter elbow 804 may further be configured to maximize the exposure area of the cuff 806, which may allow for easier cleaning and disinfecting of the cuff 806. The catheter elbow 804 may further have two or more suture points to secure it to the skin at the time the catheter is implanted, may be designed to be split and readily removed from the catheter in order to facilitate ease of removal at the end of treatment. It may be understood that once the catheter elbow 804 is removed a coring tool may the be utilized to cut the skin and soft tissue around the cuff 806 and placed over the catheter tube in order to fully remove the catheter assembly 800.

The delivery module 810 may further contain a check valve that may prevent fluid backflow. The check valve may mitigate compound, saline, or body fluids from exiting the patient through the infusion line connector during connection, removal, or non-use of the infusion line. In some embodiments the delivery module 810 may have an operation position 820, and a storage position 860. In the storage position 860 the deliver module 810 may collapse into and be secured by the retention module 812, which may decrease the profile of the delivery module 810 and prevent snagging or catching while not in use.

The retention module 814 may mitigate fluid loss out of the catheter when the infusion line is not connected, for example via the cap 812, check valve, or both. In some embodiments the cap 812 may provide visual or tactile feedback when properly placed, for example through the use of a magnet. The retention module 814 may further secure a portion of the external catheter 808 a few cm away from the pivot point or catheter elbow 804, which may provide strain relief, and in conjunction with sutures to the skin may provide strain relief to the catheter as it enters the body.

It may be understood that the catheter assembly 800 may be connected to an external pump and may be utilized to deliver treatment to an area interior to a patient, for example a washout treatment that includes cleaning an infected joint or other area without requiring removal of prosthetic hardware and/or a revision treatment such as cleaning and removal or replacement of prosthetic hardware. It may be understood the connections between the catheter, infusion line, and any external structure (such as a pump) may be liquid tight.

In some embodiments the above described systems and methods may be utilized to treat PJI. It may be understood that in other embodiments the above systems and methods may additionally be used to treat: other orthopedic infectious-disease conditions, for example osteomyelitis, septic arthritis, or fracture-related infections; surgical site infections; other implant-related infections; and/or other joint conditions.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. A catheter assembly comprising:
a catheter elbow;
a catheter that runs through the catheter elbow, the catheter including an external catheter portion on a first side of the catheter elbow and an internal catheter portion on a second side of the catheter elbow;
a cuff covering a portion of the internal catheter extending from the catheter elbow;
a delivery module attached to one end of the catheter, the delivery module configured to interface with a pump; and
a retention module configured to retain the catheter.

2. The catheter assembly of claim 1, further comprising an adhesive patch configured to adhere to a patient's skin; and
wherein the retention module is attached to the adhesive patch via one or more hook and loop pads.

3. The catheter assembly of claim 1, wherein the catheter elbow comprises;
a first catheter elbow piece; and
a second catheter elbow piece;
wherein the first catheter elbow piece and the second catheter elbow piece are configured to be combined such that a catheter may be fed through the catheter elbow.

4. The catheter assembly of claim 3, wherein the first catheter elbow piece further includes a pin and the second catheter elbow piece further includes a hole;
wherein the pin is configured to interface with the hole such that the first catheter elbow piece and the second catheter elbow piece are joined together.

5. The catheter assembly of claim 3, wherein the first catheter elbow piece further includes a pin and the second catheter elbow piece are joined together via a retaining ring or an adhesive.

6. The catheter assembly of claim 3, wherein the catheter elbow includes a groove that allows for a tool to be used to split the catheter elbow into the first catheter elbow piece and the second catheter elbow piece.

7. The catheter assembly of claim 3, wherein the catheter elbow includes two or more suture points configured to secure the catheter elbow to a patient's skin.

8. The catheter assembly of claim 1, wherein the delivery module is configured to interface with a pump via a Luer-lock; and
the pump is configured to deliver a drug and/or saline formulation through the catheter via the delivery module.

9. The catheter assembly of claim 8, further comprising an infusion line that connects the pump to the external catheter; and
a cap that covers the delivery module when the delivery module is not connected to the pump.

10. The catheter assembly of claim 9, wherein the cap provides tactile and audio feedback when secured to the delivery module.

11. The catheter assembly of claim 10, wherein the cap includes a magnet.

12. The catheter assembly of claim 9, wherein the delivery module further comprises a check valve that prevents backflow through the catheter.

13. The catheter assembly of claim 9, wherein the infusion line forms a liquid tight connection between the pump and the catheter.

14. The catheter assembly of claim 8, wherein the delivery module may alternate between a stored position in the retention module and a deployed position when interfaced with the pump.

15. The catheter assembly of claim 1, wherein the cuff is comprised of a polyester material such as Dacron, and/or metal mesh materials such as titanium or stainless steel.

16. The catheter assembly of claim 3, wherein the catheter elbow exposes the cuff to allow easy swabbing.

17. The catheter assembly of claim 1, wherein the external catheter portion is glued to the first side of the catheter elbow and the internal catheter portion is glued to the second side of the catheter elbow.
